(19) United States
(12) Patent Application Publication
(10) Pub. No.: US 2002/0048597 A1
(43) Pub. Date: Apr. 25, 2002

(54) COSMETIC AND DERMATOLOGICAL PREPARATIONS WITH A CONTENT OF CHITOSAN AND PHOSPHOLIPIDS

(76) Inventors: RAINER KROPKE, SCHENEFELD (DE); GUNTHER SCHNEIDER, HAMBURG (DE)

Correspondence Address: KURT G. BRISCOE, NORRIS, MCLAUGHLIN & MARCUS, 220 EAST 42ND STREET, 30TH FLOOR, NEW YORK, NY, 10017, US

(21) Appl. No.: 09/376,794

(22) Filed: Aug. 18, 1999

(51) Int. Cl. A61K 9/127

(52) U.S. Cl. 424/450

(57) ABSTRACT

Cosmetic or dermatological preparations which comprise

(a) chitosan having an average molecular weight of from 10,000 to 2,000,000 g/mol and a degree of deacylation of from 10 to 99% and

(b) one or more phospholipids.

The present invention relates to cosmetic and dermatological preparations with a content of chitosan and phospholipids.

The skin is the largest human organ. Its many functions (for example heat regulation and as a sense organ) include the barrier function, which prevents the skin (and thus ultimately the organism overall) from drying out and is indeed the most important function. At the same time the skin acts as a protective device against the penetration and absorption of substances approaching from the outside. This barrier function is effected by the epidermis which, as the outermost layer, forms the actual protective sheath against the environment. Being about one tenth of the overall thickness, it is also the thinnest layer of the skin.

The epidermis is a stratified tissue in which the outer layer, the horny layer (Stratum corneum), is the part which is of significance for the barrier function. The Elias skin model, which is currently recognized in the specialist field (P.M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) are the bricks, and the lipid membrane in the intercellular spaces, which is of complex composition, corresponds to the mortar. This system is essentially a physical barrier to hydrophilic substances, but, because of its narrow and multilayered structure, can equally, however, also be passed by lipophilic substances only with difficulty.

The present invention relates, in a particular embodiment, to cosmetic or pharmaceutical preparations having a reduced feel of tackiness, to processes for their preparation, and to the use of active ingredients for reducing the feel of tackiness of cosmetic preparations.

Cosmetic skincare is predominantly taken to mean that the natural function of the skin as a barrier against environmental influences (e.g. dirt, chemicals, microorganisms) and against the loss of endogenous substances (e.g. water, natural fats, electrolytes) is strengthened or rebuilt.

If this function is impaired, increased resorption of toxic or allergenic substances or attack by microorganisms may result, leading to toxic or allergic skin reactions.

Another aim of skincare is to compensate for the loss by the skin of lipids and water caused by daily washing. This is particularly important if the natural regeneration ability is insufficient. Furthermore, skincare products should protect against environmental influences, in particular against sun and wind, and delay skin ageing.

Medical compositions generally comprise one or more medicaments in an effective concentration. For the sake of simplicity, in order to clearly distinguish between cosmetic and medicinal use and corresponding products, reference is made to the legal provisions in the Federal Republic of Germany (e.g. Cosmetics Directive, Foods and Drugs Act).

Cosmetic or dermatological preparations are frequently in the form of finely disperse multiphase systems in which one or more fatty or oily phases are present in addition to one or more aqueous phases. Of these systems, in turn, the actual emulsions are the most widespread.

Chitosan is a partially deacylated chitin. This biopolymer has, inter alia, film-forming properties and is characterized by a silky feel on the skin. A disadvantage, however, is its severe tackiness on the skin which occurs in particular—temporarily—during use. In isolated instances it is not possible to market corresponding preparations since they are unacceptable to and viewed negatively by the consumer.

It is known to reduce this feel of tackiness or also feel of greasiness by adding certain substances, for example some selected powder raw ingredient, in particular talc. However, apart from the fact that this is only rarely completely successful, such an addition also alters the viscosity of the product in question and reduces the stability.

The object was therefore to remedy all of these prior art disadvantages. In particular, the intention was to provide products with reduced tackiness and greasiness. Products in the field of care cosmetics, decorative cosmetics and pharmacological technology should likewise be freed from the described disadvantages of the prior art.

It was a further object of the invention to develop cosmetic bases for cosmetic preparations which are characterized by good tolerability by the skin.

It was a further object of the present invention to provide products which have as many diverse uses as possible. For example, the intention was to provide bases for preparation forms such as cleansing emulsions, face and bodycare preparations, but also distinctly medicinal-pharmaceutical presentations, for example preparations against acne and other skin conditions.

Chitosan is characterized by the following structural formula:

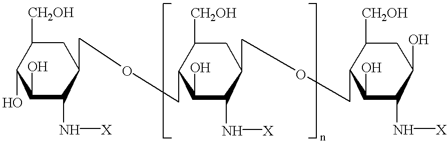

where n assumes values up to about 10,000, and X is either the acetyl radical or hydrogen. Chitosan is produced by deacetylation and partial depolymerization (hydrolysis) of chitin, which is characterized by the structural formula

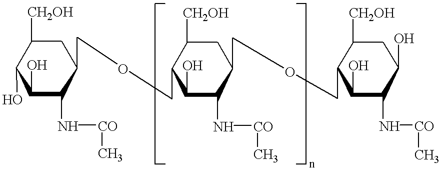

Chitin is an essential constituent of the ectoskeleton [′oχιτων=Greek: integument] of arthropods (e.g. insects, crabs, spiders) and is also found in supporting tissues of other organisms (e.g. molluscs, algae, fungi).

Chitosan is a raw material known in haircare. It is suitable, to a better degree than the chitin on which it is based, as a thickener or stabilizer and improves the adhesion and water resistance of polymeric films. A representative of a large number of literature references for the prior art is: H. P. Fiedler, “Lexikon der Hilfsstoffe für Pharmazie, Kosmetik und angrenzende Gebiete” [Lexicon of auxiliaries for pharmacy, cosmetics and related fields], third edition 1989, Editio Cantor, Aulendorf, p. 293, keyword “chitosan”.

In the region of about pH<6, chitosan is positively charged and in that range is also soluble in aqueous systems. It is incompatible with anionic raw materials. For the preparation of chitosan-containing oil-in-water emulsions, the use of nonionic emulsifiers therefore presents itself. The latter are known per se, for example from EP-A 776 657. The emulsions listed therein (e.g. with the O/W emulsifier cetylstearyl glucoside in a mixture with cetylstearyl alcohol) do, however, have certain disadvantages as regards their sensory quality.

Another aim of the invention was therefore to provide chitosan-containing preparations, in particular emulsions, in particular O/W emulsions, which are stable, can be formulated to be free-flowing or cream-like, have very good cosmetic properties, in particular as regards tackiness, and are very well tolerated by the skin and have very good skincare performance.

Surprisingly, these objects are achieved by cosmetic or dermatological preparations which comprise

(a) chitosan having an average molecular weight of from 10,000 to 2,000,000 g/mol and a degree of deacylation of from 10 to 99% and

(b) one or more phospholipids.

The preparations according to the invention are characterized by increased stability, in particular when they are in the form of emulsions, advantageously O/W emulsions. Also, the addition of chitosans and one or more phospholipids increases the stability of emulsions, in particular O/W emulsions.

The preparations according to the invention can be formulated either to be free-flowing or cream-like, have very good cosmetic properties, in particular as regards tackiness, and are very well tolerated by the skin and have very good skincare performance.

According to the invention, preference is given to chitosans having a degree of deacetylation of >25%, in particular >55 to 99% [determined by means of ¹H-NMR].

It is advantageous to choose chitosans with molecular weights between 10,000 and 2,000,000, in particular those with molecular weights between 100,000 and 1,000,000. [determined by means of gel permeation chromatography].

According to the invention, cosmetic or dermatological light protection preparations comprise from 0.01 to 50% by weight, preferably from 0.1 to 10% by weight, very particularly preferably from 0.25 to 2.5% by weight, of chitosans.

The chitosan is normally incorporated by adjusting a suspension of chitosan, in particular of micronized chitosan, in water to a pH of about 3.5-5.5 using organic or inorganic acids, a solution of the chitosan being obtained—normally by stirring. The resulting clear solutions are characterized, for example as 2% strength by weight solution of chitosan in 1.2% lactic acid (90% strength aqueous solution), by a viscosity according to Viscotester VT02 (Haake) which is in the range from 100 to 10,000 mPas, preferably from 200 to 5000 mpas.

Lactic acid can, for example, be used advantageously, although it is likewise advantageous to use other acids which produce soluble chitosan salts, such as, for example, phosphoric acid, acetic acid, ascorbic acid (the latter preferably with use of a protective gas), hydrochloric acid, glycolic acid, nitric acid, 2-pyrrolidone-5-carboxylic acid, malic acid, salicylic acid, benzoic acid.

Phospholipids are phosphoric di- or monoesters which, because of their fat-like solubility properties as a result of the lipophilic and hydrophilic components, are classed as lipids, and in the organism are, as membrane lipids, involved in the construction of layered structures, the membranes.

Phosphatidic acids are glycerol derivatives which have been esterified in the 1-sn- and 2-position with fatty acids (1-sn-position: mostly a saturated, 2-position: mostly mono- or polyunsaturated), but on atom 3-sn with phosphoric acid, and are characterized by the general structural formula

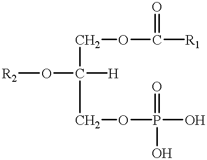

In the phosphatidic acids which occur in human or animal tissue, the phosphate radical is in most cases esterified with amino alcohols such as choline (lecithin=3-sn-phosphatidylcholine) or 2-aminoethanol (ethanolamine) or L-serine (cephalin=3-sn-phosphatidylethanolamine or sn-phosphatidyl-L-serine), with myoinositol to give the phosphoinositides [1-(3-sn-phosphatidyl)-D-myoinositols] frequent in tissues, with glycerol to give phosphatidylglycerols.

Lecithins are characterized by the general structural formula

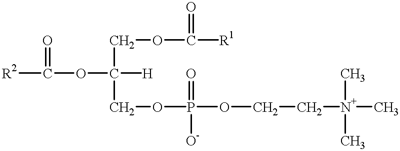

in which R and R² are typically unbranched aliphatic radicals having 15 or 17 carbon atoms and up to 4 cis-double bonds.

Cardiolipins (1,3-bisphosphatidylglycerols) are phospholipids of two phosphatidic acids linked via glycerol. Lysophospholipids are obtained when an acyl radical is cleaved off by a phospholipase A from phospholipids (e.g. lysolecithins).

Lysophospholipids are characterized by the general structural formula

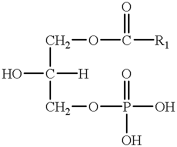

Lysolecithins, for example, are characterized by the general structural formula

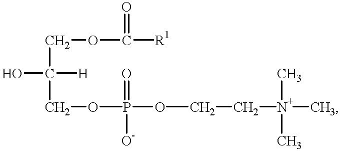

where R and R² are typically unbranched, aliphatic radicals having 15 or 17 carbon atoms and up to 4 cis-double bonds.

Phospholipids also include plasmalogens in which an aldehyde (in the form of an enol ether) is bonded in the 1-position instead of a fatty acid; the O-1 -sn-alkenyl compounds corresponding to the phosphatidylcholines are called, for example, phosphatidalcholines.

Phosphosphingolipids are based on the basic structure of sphingosine or else phytosphingosine, which are characterized by the following structural formulae:

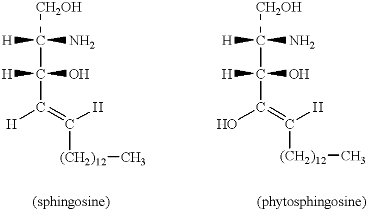

Modifications of sphingolipids are characterized, for example, by the general basic structure

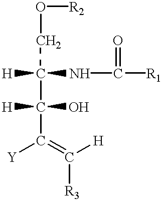

in which R₁ and R₃ independently of one another are saturated or unsaturated, branched or unbranched alkyl radicals having from 1 to 28 carbon atoms, R₂ is chosen from the group: hydrogen atom, saturated or unsaturated, branched or unbranched alkyl radicals of from 1 to 28 carbon atoms, sugar radicals, phosphate groups which are unesterified or esterified with organic radicals, sulphate groups which are unesterified or esterified with organic radicals, and Y is either a hydrogen atom, a hydroxyl group or another heterofunctional radical.

Sphingophospholipids:

[chemical structure]

R₁ and R₃ are alkyl radicals, and R₄ is an organyl radical.

Sphingomyelins are organophosphorylated sphingolipids of the type

[chemical structure]

Preferred phospholipids are lecithins. Types of lecithin which are to be used advantageously are chosen from crude lecithins, which have been deoiled and/or fractionated and/or spray-dried and/or acetylated and/or hydrolysed and/or hydrogenated.

Phospholipids to be used advantageously according to the invention are, for example, commercially available under the trade names Phospholipon 25 (Nattermann), Emulmetik 120 (Lucas Meyer), Sternpur E (Stern), Sternpur PM (Stern), Nathin 3KE (Stern).

The amount of phospholipids (one or more compounds) in the preparations is preferably from 0.001 to 30% by weight, particularly preferably 0.05-20% by weight, in particular 0.5-5% by weight, based on the total weight of the preparation.

According to the invention, it is possible and advantageous to choose freely the content of the oily phase in the preparations according to the invention in the range from 0.01 to 99% by weight, based on the total weight of the preparations.

Base constituents of the preparations according to the invention which can be used are:

water or aqueous solutions

aqueous ethanolic solutions

natural oils and/or chemically modified natural oils and/or synthetic oils;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used.

For the purposes of the present invention, the oily phase of the emulsions is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, and from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl. myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oily phase can also be chosen advantageously from the group of branched and unbranched hydrocarbons and hydrocarbon waxes, silicon oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglyercides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Fatty and/or wax components which are to be used advantageously according to the invention can be chosen from the group of vegetable waxes, animal waxes, mineral waxes and petrochemical waxes. Examples which are favourable according to the invention are candelilla wax, carnauba wax, japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes.

Other advantageous fatty and/or wax components are chemically modified waxes and synthetic waxes, such as, for example, those obtainable under the trade names Syncrowax HRC (glyceryl tribehenate), Syncrowax HGLC (C₁₆₋₃₆ fatty acid triglyceride) and Syncrowax AW 1C (C₁₈₋₃₆ fatty acid) from CRODA GmbH, and montan ester waxes, Sasol waxes, hydrogenated jojoba waxes, synthetic or modified beeswaxes (e.g. dimethicone copolyol beeswax and/or C₃₀₋₅₀ alkyl beeswax), polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, trihydroxystearin, fatty acids, fatty acid esters, and glycol esters, such as, for example, C₂₀₋₄₀-alkyl stearate, C₂₀₋₄₀-alkylhydroxystearoyl stearate and/or glycol montanate. Also advantageous are certain organosilicon compounds, which have similar physical properties to the specified fatty and/or wax components, such as, for example, stearoxytrimethylsilane.

According to the invention, the fatty and/or wax components can be present either individually or as a mixture.

Any desired mixtures of such oil and wax components can also be used advantageously for the purposes of the present invention. In some instances, it can also be advantageous to use waxes, for example cetyl palmitate, as the sole lipid component of the oily phase.

The oily phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene can be used advantageously for the purposes of the present invention.

The oily phase can advantageously additionally have a content of cyclic or linear silicone oils, or consist entirely of such oils, although it is preferable to use an additional content of other oily phase components apart from the silicone oil or the silicone oils.

Cyclomethicone (octamethylcyclotetrasiloxane) can be used advantageously as silicon oil to be used according to the invention. However, other silicon oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

For the purposes of the present invention, emulsions according to the invention, for example in the form of a skin protection cream, a skin lotion, a cosmetic milk, for example in the form of a sun protection cream or a sun protection milk, are advantageous and comprise, for example, fats, oils, waxes and/or other fatty substances, and water and one or more emulsifiers as are customarily used for this type of formulation.

The person skilled in the art is of course aware that demanding cosmetic compositions are in most cases inconceivable without the customary auxiliaries and additives. These include, for example, bodying agents, fillers, perfume, dyes, emulsifiers, additional active ingredients such as vitamins or proteins, light protection agents, stabilizers, insect repellents, alcohol, water, salts, antimicrobial, proteolytic or keratolytic substances, etc.

Corresponding requirements apply mutatis mutandis to the formulation of medicinal preparations.

For the purposes of the present invention, medicinal topical compositions generally comprise one or more medicaments in an effective concentration. For the sake of simplicity, in order to distinguish clearly between cosmetic and medicinal use and corresponding products, reference is made to the legal provisions in the Federal Republic of Germany (for example Cosmetics Directive, Foods and Drugs Act).

Accordingly, for the purposes of the present invention, cosmetic or topical dermatological compositions can, depending on their composition, be used for example as skin protection cream, cleansing milk, sunscreen lotion, nourishing cream, day or night cream, etc. If desired, it is possible and advantageous to use the compositions according to the invention as a base for pharmaceutical formulations.

It is likewise advantageous to make use of the properties according to the invention in the form of decorative cosmetics (make-up formulations).

Those cosmetic and dermatological preparations which are in the form of a sunscreen are also favourable. In addition to the active ingredient used according to the invention, these also preferably comprise at least one UVA filter substance and/or at least one UVB filter substance and/or at least one inorganic pigment.

However, it is also advantageous for the purposes of the present invention to provide cosmetic and dermatological preparations whose main purpose is not protection against sunlight, but which nevertheless still contain anti-UV substances. Thus, for example, UV-A and UV-B filter substances are usually incorporated in day creams.

Preparations according to the invention can advantageously comprise substances which absorb UV radiation in the UVB region, the total amount of filter substances being, for example, from 0.1% by weight to 30% by weight, preferably from 0.5 to 10% by weight, in particular from 1 to 6% by weight, based on the total weight of the preparations.

The UVB filters can be oil-soluble or water-soluble. Examples of oil-soluble substances which may be mentioned are:

3-benzylidenecamphor and derivatives thereof, e.g. 3-(4-methylbenzylidene)camphor,

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzo- phenone;

esters of benzalmalonic acid, preferably di(2-ethylhexyl) 4-methoxybenzalmalonate;

2,4,6-trianilino(p-carbo-2′-ethyl-1′-hexyloxy)-1,3,5-triazine.

Advantageous water-soluble substances are:

2-phenylbenzimidazole-5-sulphonic acid and salts thereof, for example sodium, potassium or triethanolammonium salts,

sulphonic acid derivatives of benzophenones, preferably 2-hydroxy-4-methoxybenzophenone-5-sulphonic acid and its salts;

sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)-sulphonic acid and its salts.

The list of given UVB filters which can be used according to the invention is of course not intended to be limiting.

It can also be advantageous to use UVA filters that are usually present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)-propane-1,3-dione. Preparations which comprise these combinations are also provided by the invention. It is possible to use the same amounts of UVA filter substances which were given for UVB filter substances,

For the purposes of the present invention, cosmetic and/or dermatological preparations can also comprise inorganic pigments which are usually used in cosmetics for protecting the skin against UV radiation. These are oxides of titanium, zinc, iron, zirconium, silicon, manganese, aluminium, cerium and mixtures thereof, and modifications in which the oxides are the active agents. Particular preference is given to pigments based on titanium dioxide. It is possible to use the quantities given for the above combinations.

The cosmetic and dermatological preparations according to the invention can comprise cosmetic active ingredients, auxiliaries and/or additives as are usually used in such preparations, for example antioxidants, preservatives, bactericides, perfumes, antifoams, dyes, pigments which have a coloring effect, thickeners, surfactants, emulsifiers, emollients, moisturizers and/or humectants, fats, oils, waxes or other usual constituents of a cosmetic or dermatological formulation, such as alcohols, polyols, polymers, foam stabilizers, electrolytes, organic solvents or silicone derivatives.

For the purposes of the present invention, it is advantageous to add other anti-irritative or anti-inflammatory active ingredients to the preparations, in particular batyl alcohol (α-octadecyl glyceryl ether), selachyl alcohol (α-9-octadecenyl glyceryl ether), chimyl alcohol (α-hexadecyl glyceryl ether), bisabolol and/or panthenol.

It is likewise advantageous to add conventional antioxidants to the preparations for the purposes of the present invention. According to the invention, favourable antioxidants can be any antioxidants which are suitable or customary for cosmetic and/or dermatological applications.

The antioxidants are advantageously selected from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, χ-lycopene) and derivatives thereof, chlorogenic acid and derivatives thereof, lipoic acid and derivatives thereof (e.g. dihydrolipoic acid), aurothioglucase , propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (e.g. buthionine sulphoximines, homocysteine sulphoximine, buthionine sulphones, penta-, hexa-, heptathionine sulphoximine) in very small tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin, rutinic acid and derivatives thereof, α-glucosylrutin, ferulic acid, furfurylideneglucitol, carnosine, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof are used as the antioxidant(s), it is advantageous to choose their respective concentrations from the range of 0.001-10% by weight, based on the total weight of the formulation.

The preparations according to the present invention can also be used as bases for cosmetic or dermatological deodorants or antiperspirants. All active ingredients which are common for deodorants or antiperspirants can be used advantageously, for example odour maskers such as the customary perfume constituents, odour absorbers, for example the phyllosilicates described in laid-open patent specification DE-P 40 09 347, and of these, in particular, montmorillonite, kaolinite, ilite, beidellite, nontronite, saponite, hectorite, bentonite, smectite, and also, for example, zinc salts of ricinoleic acid.

Antibacterial agents are likewise suitable for incorporation into the preparations according to the invention. Advantageous substances are, for example, 2,4,4′-trichloro-2′-hydroxydiphenyl ether (Irgasan), 1,6-di-(4-chlorophenylbiguanido)hexane (chlorhexidine), 3,4,4′-trichlorocarbanilide, quaternary ammonium compounds, oil of cloves, mint oil, oil of thyme, triethyl citrate, farnesol (3,7,11-trimethyl-2,6,10-dodecatrien-1-ol) and the active ingredients or active ingredient combinations described in laid-open patent specifications DE-37 40 186, DE-39 38 140, DE-42 04 321, DE-42 29 707, DE-43 09 372, DE-44 11 664, DE-1 95 41 967, DE-1 95 43 695, DE-1 95 43 696, DE-195 47 160, DE-196 02 108, DE-1 96 02 110, DE-1 96 02 111, DE-196 31 003, DE-1 96 31 004 and DE-1 96 34 019 and the patent specifications DE-42 29 737, DE-42 37 081, DE-43 24 219, DE-44 29 467, DE-44 23 410 and DE-1 95 16 705. Sodium hydrogencarbonate can also be used advantageously.

The amount of such active ingredients (one or more compounds) in the preparations according to the invention is preferably from 0.001 to 30% by weight, particularly preferably 0.05-20% by weight, in particular 1-10% by weight, based on the total weight of the preparation.

For the purposes of the present invention, suitable propellants for cosmetic and/or dermatological preparations which can be sprayed from aerosol containers are the customary known readily volatile, liquefied propellants, for example hydrocarbons (propane, butane, isobutane), which can be employed alone or in mixtures of one another. Compressed air can also be used advantageously.

The person skilled in the art is of course aware that there are propellant gases which are nontoxic per se and would be suitable in principle for realizing the present invention in the form of aerosol preparations, but which nevertheless should be omitted because of an unacceptable impact on the environment or other accompanying circumstances, in particular fluorinated hydrocarbons and chlorofluorocarbons (CFCs).

For the purposes of the present invention, cosmetic preparations for the treatment and care of hair which comprise the active ingredient used according to the invention can be in the form of emulsions which are of the nonionic or anionic type. Nonionic emulsions comprise, in addition to water, oils or fatty alcohols, which, for example, can also be polyethoxylated or polypropoxylated, or else mixtures of the two organic components. These emulsions optionally comprise cationic surface-active substances.

For the purposes of the present invention, the aqueous phase of the cosmetic preparations can also have gel character which, in addition to an effective content of the substances used according to the invention and the solvents used customarily therefor, preferably water, also comprises other organic thickeners, e.g. gum arabic, xanthan gum, sodium alginate, starch and starch derivatives (e.g. distarch phosphate), cellulose, cellulose derivatives, preferably methylcellulose, hydroxymethylcellulose, hydroxyethyl-cellulose, hydroxypropylcellulose, hydroxypropylmethylcellulose or inorganic thickeners, e.g. aluminium silicate such as, for example, organically modified or also unmodified hectorite, bentonite, or the like, or a mixture of polyethylene glycol and polyethylene glycol stearate or distearate. The thickener is present in the gel, for example, in an amount between 0.1 and 30% by weight, preferably between 0.5 and 15% by weight.

It can also be advantageous to add interface- or surface-active agents according to the invention to preparations, for example cationic emulsifiers such as, in particular, quaternary surfactants.

Quaternary surfactants contain at least one N-atom which is covalently bonded to 4 alkyl or aryl groups. Irrespective of the pH, this leads to a positive charge. Alkyl betaine, alkylamidopropyl betaine and alkylamidopropylhydroxysulphane are advantageous. The cationic surfactants used according to the invention can also preferably be chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethyl-ammonium chlorides or bromides, alkylamidoethyltrimethylammonium ether sulphate, alkylpyridinium salts, for example lauryl- or cetylpyridinium chloride, imidazolin derivates and compounds having a cationic character such as amine oxides, for example alkyldimethylamine oxides or alkylaminoethyldimethylamine oxide. In particular, cetyltrimethylammonium salts are to be used advantageously.

It is also advantageous to use cationic polymers (e.g. Jaguar C 162 [hydroxypropyl, Guar hydroxypropyltrimonium chloride] or modified magnesium aluminium silicates (e.g. quaternium-18 hectorite, which is obtainable, for example, under the trade name Bentone® 38 from Rheox, or stearalconium hectorite, which is obtainable, for example, under the trade name Softisan® gel from Hüls AG).

Preparations according to the invention can advantageously also comprise oil thickeners in order to improve the tactile properties of the emulsion and the stick consistency. Advantageous oil thickeners for the purposes of the present invention are, for example, other solids, such as, for example, hydrophobic silicon oxides of the Aerosil® type, which are obtainable from Degussa AG. Advantageous Aerosil® products are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974 and/or Aerosil® R976.

In addition, so-called metal soaps (i.e. the salts of higher fatty acids with the exception of the alkali metal salts) are also advantageous oil thickeners for the purposes of the present invention, such as, for example, aluminium stearate, zinc stearate and/or magnesium stearate.

It is likewise advantageous to add amphoteric or zwitterionic surfactants (e.g. cocoamidopropylbetaine) and moisturizers (e.g. betaine) to the preparations according to the invention. Examples of amphoteric surfactants which are to be used advantageously are acyl-/dialkylethylenediamine, for example sodium acylamphoacetate, disodium acylamphodipropionate, disodium alkylamphodiacetate, sodium acylamphohydroxypropylsulphonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

The amount of interface- or surface-active substances (one or more compounds) in the preparations according to the invention is preferably from 0.001 to 30% by weight, particularly preferably from 0.05-20% by weight, in particular 1-10% by weight, based on the total weight of the preparation.

Preparations according to the invention can also comprise active ingredients (one or more compounds) which are chosen from the group: acetylsalicylic acid, atropine, azulene, hydrocortisone and derivatives thereof, e.g. hydrocortisone-17 valerate, vitamins, e.g. ascorbic acid and derivatives therof, vitamins of the B and D series, very favourably vitamin B₁, vitamin B₁₂ and vitamin D₁, but also bisabolol, unsaturated fatty acids, namely the essential fatty acids (often also called vitamin F), in particular γ-linolenic acid, oleic acid, eicosapentanoic acid, docosahexanoic acid and derivatives thereof, chloramphenicol, caffeine, prostaglandins, thymol, camphor, extracts or other products of a vegetable or animal origin, e.g. evening primrose oil, starflower oil or currant seed oil, fish oils, cod-liver oil or also ceramides and ceramide-like compounds, etc. It is also advantageous to choose the active ingredients from the group of refatting substances, for example Purcellin oil, Eucerit® and Neocerit®.

The amount of such active ingredients (one or more compounds) in the preparations according to the invention is preferably from 0.001 to 30% by weight, particularly preferably from 0.05-20% by weight, in particular 1-10% by weight, based on the total weight of the preparation.

The examples below serve to illustrate the present invention.

Chitosan solution preparation example:

| | % by weight |
|---|---|
| Chitosan | 2.0 |
| Lactic acid (90% strength aqueous solution) | 1.2 |
| Water | ad 100.00 |
| pH ca. | 4.5 |

### EXAMPLE 1 (O/W emulsion)

| | % by weight |
|---|---|
| Sunflower seed oil | 5.00 |
| Chitosan solution acc. to the preparation example | 50.00 |
| Lecithin | 1.00 |
| Glycerol | 3.00 |
| Perfume, preservatives, dyes, antioxidants | q.s. |
| Water | ad 100.00 |
| pH | ca. 4.5 |

### EXAMPLE 2 (O/W emulsion)

| | % by weight |
|---|---|
| Wheatgerm oil | 5.0 |
| Chitosan solution acc. to the preparation example | 50.0 |
| Lecithin | 1.0 |
| Distarch phosphate | 1.0 |
| Glycerol | 3.0 |
| Perfume, preservatives, dyes, antioxidants | q.s. |
| pH | ca. 4.5 |
| Water | ad. 100.0 |

### EXAMPLE 3 (O/W emulsion)

| | % by weight |
|---|---|
| Jojoba oil | 3.00 |
| Caprylic/capric triglycerides | 3.00 |
| Dimethicone | 0.50 |
| Dimethiconol | 0.10 |
| Cyclomethicone | 2.00 |
| Dimethicone copolyol | 0.20 |
| Chitosan solution acc. to the preparation example | 50.00 |
| Lecithin | 2.00 |
| Tocopherol acetate | 0.50 |
| Panthenol | 0.50 |
| Biotin | 0.10 |
| Glycerol | 3.00 |
| 1,3-Butylene glycol | 1.50 |
| Serine | 0.20 |
| Perfume, preservatives, dyes, antioxidants | q.s. |
| Water | ad 100.00 |
| pH | ca. 4.5 |

### EXAMPLE 4 (O/W emulsion)

| | % by weight |
|---|---|
| Dimethicone | 2.00 |
| Cyclomethicone | 2.00 |
| Chitosan solution acc. to the preparation example | 50.00 |
| Lecithin | 1.50 |
| Distarch phosphate | 1.00 |
| Glycerol | 3.00 |
| Betaine | 2.00 |
| Perfume, preservatives, dyes, antioxidants | q.s. |
| Water | ad 100.00 |
| pH | ca. 4.5 |

### EXAMPLE 5 (O/W emulsion)

| | % by weight |
|---|---|
| Sunflower seed oil | 12.00 |
| Chitosan solution acc. to the preparation example | 75.00 |
| Lecithin | 4.00 |
| Glycerol | 3.00 |
| Perfume, preservatives, dyes, antioxidants | q.s. |
| Water | ad 100.00 |
| pH | Ca. 4.5 |

### EXAMPLE 6 (O/W emulsion)

| | % by weight |
|---|---|
| Safflor oil | 5.00 |
| Chitosan solution acc. to the preparation example | 50.00 |
| Lecithin | 1.00 |
| Glycerol | 5.00 |
| Distarch phosphate | 1.00 |
| Perfume, preservatives, dyes, antioxidants | q.s. |
| Water | ad 100.00 |
| pH | ca. 4.5 |

### EXAMPLE 7 (O/W emulsion)

| | % by weight |
|---|---|
| Chitosan solution acc. to the preparation example | 50.00 |
| Octyldodecanol | 2.00 |
| Squalane | 2.00 |
| Paraffinum liquidum | 1.00 |
| Hydrogenated polyisobutene | 1.00 |
| Lecithin | 2.00 |
| 4-(tert-Butyl)-4′-methoxydibenzoylmethane | 2.00 |
| Octyl methoxycinnamate | 2.50 |
| 4-Methylbenzylidenecamphor | 2.50 |
| Tris[anilino(p-carbo-2′-ethyl-1′-hexyloxy)]triazine | 1.50 |
| Titanium dioxide | 2.00 |
| Glycerol | 3.00 |
| Perfume, preservatives, dyes, antioxidants | q.s. |
| Water | ad 100.00 |
| pH | ca. 4.5 |

### EXAMPLE 8 (O/W emulsion)

| | % by weight |
|---|---|
| Chitosan solution acc. to the preparation example | 50.00 |
| Octyldodecanol | 1.00 |
| C₁₂₋₁₅-Alkyl benzoate | 1.00 |
| Squalane | 1.00 |
| Lecithin | 1.50 |
| Glycerol | 3.00 |
| Perfume, preservatives, dyes, antioxidants | q.s. |
| Water | ad 100.00 |
| pH | ca. 4.5 |

### EXAMPLE 9 (O/W emulsion)

| | % by weight |
|---|---|
| Chitosan solution acc. to the preparation example | 50.00 |
| Octyldodecanol | 1.00 |
| Dacaprylyl ether | 1.00 |
| Squalane | 1.00 |
| Paraffinum liquidum | 1.00 |
| Hydrogenated polyisobutene | 1.00 |
| Glyceryl stearate citrate | 0.40 |
| Cetylstearyl alcohol | 0.20 |
| Lecithin | 1.50 |
| Glycerol | 3.00 |
| Perfume, preservatives, dyes, antioxidants | q.s. |
| Water | ad 100.00 |
| pH | ca. 4.5 |

### EXAMPLE 1 0 (O/W emulsion)

| | % by weight |
|---|---|
| Chitosan solution acc. to the preparation example | 50.00 |
| Distearyldimethylammonium chloride | 2.00 |
| Squalane | 2.00 |
| Paraffinum liquidum | 2.00 |
| Hydrogenated polyisobutene | 2.00 |
| Lecithin | 1.50 |
| Glycerol | 3.00 |
| Perfume, preservatives, dyes, antioxidants | q.s. |
| Water | ad |
| | 100.00 |
| pH | ca. |
| | 4.5 |

### EXAMPLE 1 1 (W/O emulsion)

| | % by weight |
|---|---|
| PEG-7-hydrogenated castor oil | 4.00 |
| Wool wax alcohol | 1.50 |
| Chitosan solution acc. to the preparation example | 0.20 |
| Squalane | 1.00 |
| Lecithin | 0.20 |
| Beeswax | 3.00 |
| Vaseline | 4.00 |
| Ozokerite | 4.00 |
| Paraffin oil, subliquidum | 8.00 |
| 4-(tert-Butyl)-4′-methoxydibenzoylmethane | 2.00 |
| Octylmethoxycinnamate | 2.50 |
| 4-Methylbenzylidenecamphor | 2.50 |
| Tris[anilino-(p-carbo-2′-ethyl-1′-hexyloxy)]triazin | 1.50 |
| Titanium dioxide | 2.00 |
| Tocopherol acetate | 1.00 |
| Glycerol | 3.00 |
| Sodium chloride | 0.70 |
| Perfume, preservatives, dyes, antioxidants | q.s. |
| Water | ad |
| | 100.00 |

### EXAMPLE 12 (W/O emulsion)

| | % by weight |
|---|---|
| Polyglyceryl-3 dioleate | 3.50 |
| Ozokerite | 3.00 |
| Beeswax | 2.00 |
| Paraffin oil, subliquidum | 10.00 |
| Cetylstearyl octanoate | 10.00 |
| Chitosan solution acc. to the preparation example | 20.00 |
| Lecithin | 0.40 |
| Glycerol | 5.00 |
| Sodium chloride | 0.70 |
| Perfume, preservatives, dyes, antioxidants | q.s. |
| Water | ad |
| | 100.00 |

### EXAMPLE 13 (W/O emulsion)

| | % by weight |
|---|---|
| Laurylmethicone copolyol | 1.50 |
| Cetylmethicone copolyol | 0.50 |
| Paraffin oil, subliquidum | 20.00 |
| Cylomethicone | 2.00 |
| Dimethicone | 5.00 |
| Chitosan solution acc. to the preparation example | 10.00 |
| Lecithin | 0.20 |
| Glycerol | 10.00 |
| Sodium chloride | 1.00 |
| Perfume, preservatives, dyes, antioxidants | q.s. |
| Water | ad |
| | 100.00 |

### EXAMPLE 14 (“hydrodispersion”)

| | % by weight |
|---|---|
| Sunflower seed oil | 2.50 |
| Chitosan solution acc. to the preparation example | 50.00 |
| Lecithin | 0.10 |
| Distarch phosphate | 1.00 |
| Hectorite | 0.50 |
| Cellulose glycolate | 0.50 |
| Glycerol | 3.00 |
| Perfume, preservatives, dyes, antioxidants | q.s. |
| Water | ad |
| | 100.00 |
| pH | ca. |
| | 4.5 |

### EXAMPLE 15 (“hydrodispersion”):

| | % by weight |
|---|---|
| Sunflower seed oil | 2.50 |
| Chitosan solution acc. to the preparation example | 50.00 |
| Lecithin | 0.10 |
| Distarch phosphate | 1.00 |
| Tromethamine magnesium aluminium silicate | 0.50 |
| Cellulose glycolate | 0.50 |
| Glycerol | 3.00 |
| Perfume, preservatives, dyes, antioxidants | q.s. |
| Water | ad |
| | 100.00 |
| pH | ca. |
| | 4.5 |

### EXAMPLE 16 (emulsions make-up)

| | % by weight |
|---|---|
| Sunflower seed oil | 7.50 |
| Chitosan solution acc. to the preparation example | 50.00 |
| Lecithin | 2.00 |
| Distarch phosphate | 1.00 |
| Dimethicone | 0.50 |
| Glycerol | 1.50 |
| Magnesium silicate | 1.00 |
| Mica | 1.00 |
| Iron oxides | 1.00 |
| Titanium dioxide | 2.50 |
| Talc | 5.00 |
| Tapioca starch | 0.25 |
| Perfume, preservatives, dyes, antioxidants | q.s. |
| Water | ad |
| | 100.00 |
| pH | ca. |
| | 4.5 |

### EXAMPLE 17 (emulsion lipcare stick)

| | % by weight |
|---|---|
| Octyldodecanol | 20.00 |
| Polyglyceryl-3 dioleate | 3.50 |
| Beeswax | 12.50 |
| Squalane | 11.00 |
| C₂₀₋₄₀-Alkyl stearates | 5.00 |
| Jojoba oil | 10.00 |
| Carnauba wax | 2.00 |
| Tocopherol acetate | 0.70 |
| Chitosan solution acc. to the preparation example | 5.00 |
| Lecithin | 1.00 |
| Perfume, preservatives, dyes, antioxidants | q.s. |
| Caprylic acid/capric triglycerides | ad |
| | 100.0 |

### EXAMPLE 18 (alcoholic face tonic)

| | % by weight |
|---|---|
| Chitosan solution acc. to the preparation example | 50.00 |
| Lecithin | 0.10 |
| Ethanol | ad |
| | 100.00 |

### EXAMPLE 19 (O/W emulsion)

| | % by weight |
|---|---|
| Soya oil | 5.00 |
| Chitosan solution acc. to the preparation example | 50.00 |
| Lecithin | 1.00 |
| Hydroxypropylmethylcellulose | 0.50 |
| Ethanol | 5.00 |
| Perfume, preservatives, dyes, antioxidants | q.s. |
| Water | ad |
| | 100.00 |
| pH | ca. |
| | 4.5 |

### EXAMPLE 20 (O/W emulsion)

| | % by weight |
|---|---|
| Soya oil | 5.00 |
| Chitosan solution acc. to the preparation example | 50.00 |
| Lecithin | 1.00 |
| Isostearyl phosphate | 0.25 |
| Ammonium phosphatide | 0.25 |
| Perfume, preservatives, dyes, antioxidants | q.s. |
| Water | ad. 100.00 |
| pH | ca. 4.5 |

Comparison of the formulations containing combinations of chitosan and phospholipids (here: lecithin) used according to the invention with formulations which comprise only one of the constituents chitosan and phospholipids shows that the combinations used according to the invention lead to increased stabillity of the underlying emulsions (here: O/W emulsions).

### O/w emulsion as in example 1

| | % by weight | | |
|---|---|---|---|
| Sunflower seed oil | 5.00 | 5.00 | 5.00 |
| Chitosan solution acc. to the | 50.00 | — | 50.00 |
| preparation example | | | |
| Lecithin | 1.00 | 1.00 | — |
| Glycerol | 3.00 | 3.00 | 3.00 |
| Perfume, preservatives, dyes, | q.s. | q.s. | q.s. |
| antioxidants | | | |
| Water | ad 100.00 | ad 100.00 | ad 100.00 |
| pH | 4.5 | 4.5 | 4.5 |
| Stability of the emulsion: | stable | unstable | unstable |

1. Cosmetic or dermatological preparations which comprise
(a) chitosan having an average molecular weight of from 10,000 to 2,000,000 g/mol and a degree of deacylation of from 10 to 99% and
(b) one or more phospholipids.

2. Use of combinations of
(a) chitosan having an average molecular weight of from 10,000 to 2,000,000 g/mol and a degree of deacylation of from 10 to 99% and
(b) one or more phospholipids for the preparation of non-tacky cosmetic preparations, in particular O/W emulsions, or for reducing the tackiness of cosmetic preparations, in particular O/W emulsions.

3. Use of combinations of
(a) chitosan having an average molecular weight of from 10,000 to 2,000,000 g/mol and a degree of deacylation of from 10 to 99% and
(b) one or more phospholipids for increasing the stability of cosmetic preparations, in particular O/W emulsions.
